(19) United States
(12) Patent Application Publication
(10) Pub. No.: US 2014/0243579 A1
(43) Pub. Date: Aug. 28, 2014

(54) DUAL-ENERGY IMAGE SUPPRESSION METHOD

(71) Applicant: Loyola University Chicago, Maywood, IL (US)

(72) Inventors: John C. Roeske, Naperville, IL (US); Jason P. Luce, Ann Arbor, MI (US); Tracy Sherertz, Cleveland, OH (US)

(73) Assignee: Loyola University Chicago, Maywood, IL (US)

(21) Appl. No.: 14/191,991

(22) Filed: Feb. 27, 2014

Related U.S. Application Data
(60) Provisional application No. 61/769,961, filed on Feb. 27, 2013.

(51) Int. Cl. A61N 5/10; G06T 7/00; A61B 6/00

(52) U.S. Cl. CPC A61N 5/1039; A61B 6/485; G06T 7/0012
USPC 600/1; 382/132

(57) ABSTRACT

An imaging process capable of selectively enhancing visualization of soft tissues, for example, a tumor. The imaging process includes producing a hard tissue-enhanced image of a body containing both soft and hard tissues, wherein the hard tissue-enhanced image contains images of the hard tissues that are enhanced relative to the soft tissues. A radiographic image of the body and the soft and hard tissues thereof are then obtained, after which a weighted subtraction algorithm is performed between the radiographic image and the hard tissue-enhanced image to produce a soft tissue-enhanced image in which imaging of the soft tissues is enhanced relative to the hard tissues. The hard tissue-enhanced image may be produced by obtaining first and second initial radiographic images of the body including the soft and hard tissues, and then performing a weighted subtraction algorithm on the first and second initial radiographic images to produce the hard tissue-enhanced image.

### CROSS REFERENCE TO RELATED APPLICATIONS

This application claims the benefit of U.S. Provisional Application No. 61/769,961, filed Feb. 27, 2013, the contents of which are incorporated herein by reference.

### BACKGROUND OF THE INVENTION

The present invention generally relates to the use of dual-energy (DE) imaging techniques for therapeutic and/or diagnostic purposes. More particularly, this invention relates to a technique for using dual-energy imaging techniques to selectively highlight and/or enhance the visualization of certain tissue, nonlimiting examples of which include soft tissues such as lung tumors, and the ability to use such visualization in therapies, a nonlimiting example of which is image-guided radiotherapy.

Various diagnostic and therapy techniques make use of imaging that involves highlighting or enhancing the visualization of soft tissues by suppressing the imaging of hard tissues, typically bone when attempting to visualize surrounding soft tissues. A particular example utilized to visualize soft tissues in the rib cage entails rib suppression utilizing a dual-energy (DE) imaging technique. Briefly, and as represented by a flowchart in FIG. 1, this technique involves obtaining a radiograph (“Image 1 High Energy”) at a relatively high energy (for example, 120 kVp) and another radiograph (“Image 1 Low Energy”) at a lower energy (for example, 60 kVp). A third image (“Image 1 DE Soft Tissue”) can then be created that highlights or enhances soft tissue by performing a weighted subtraction of the logarithm of these images. FIG. 2 represents this process, which shows two radiographic images (left and center) produced at, respectively, high energy (120 kVp) and low energy (60 kVp), and a third image (“DE Soft”) generated by the identified logarithmic subtraction step. As evident from these images, the high energy image enhances the image of soft tissue (the spherical object in FIG. 2), the low energy image enhances the image of hard tissue (the ribs in FIG. 2), and the logarithmic subtraction step results in enhanced visualization of the image of the spherical object in the third image as a result of the image of the ribs being suppressed. As indicated in FIG. 1, the creation of each desired “DE Soft” image entails the acquisition of both high-energy and low-energy images, on which a logarithmic subtraction step can be performed to create the “DE Soft” image.

The use of dual-energy imaging from a single image has also been investigated, particularly by the use of a massively trained neural network (MTNN) technique. The MTNN is trained using a database of radiographs such that when a new radiograph is considered, the program will extract the soft tissue image. In addition, methods for extracting the ribs from a single chest radiograph have also been developed. The ribs can then be added back to the image (using a different weighting) to create a virtual image with an effective energy different from the first. Dual-energy imaging subtraction is then used to suppress the bone and highlight soft tissue.

Various shortcomings have been associated with the use of prior dual-energy imaging techniques. Because dual-energy imaging involves the use of two radiographs, the quality of the third image generated by logarithmic subtraction can be degraded as a result of cardiac and respiratory motion that occurs between the acquisition of the two original images. This motion results in artifacts that can blur the overall image and, in particular, soft image such as a tumor that a physician is trying to detect with the imaging technique. In addition, the generation of a single image requires the acquisition of two (high and low energy) radiographs, each subjecting a patient to an additional dose of radiation. Though potentially entailing fewer radiation exposures, MTNN methods are computationally intensive and require a large database to train the neural network. In addition, MTNN methods are generally limited to anterior/posterior radiographs because radiographs obtained at oblique angles typically result in the incomplete subtraction of the ribs or other hard tissue.

Accordingly, there is an ongoing need for techniques capable of creating highlighted or enhanced images of soft tissues.

### BRIEF DESCRIPTION OF THE INVENTION

The present invention provides an imaging process suitable for selectively highlighting and/or enhancing the visualization of soft tissues, for example, a tumor, and which may be used in therapies, for example, image-guided radiotherapy.

According to an aspect of the invention, an imaging process includes producing a hard tissue-enhanced image of a body containing both soft and hard tissues, wherein the hard tissue-enhanced image contains images of the hard tissues that are enhanced relative to the soft tissues. A radiographic image of the body and the soft and hard tissues thereof are then obtained, after which a weighted subtraction algorithm is performed between the radiographic image and the hard tissue-enhanced image to produce a soft tissue-enhanced image in which imaging of the soft tissues is enhanced relative to the hard tissues. According to an optional but preferred aspect, the hard tissue-enhanced image is produced by obtaining first and second initial radiographic images of the body and the soft and hard tissues thereof, and then performing a weighted subtraction algorithm on the first and second initial radiographic images to produce the hard tissue-enhanced image.

Other aspects of the invention include diagnostic and therapeutic techniques that make use of a hard tissue-enhanced image acquired by a process comprising the steps described above. A particular but nonlimiting example is image-guided radiotherapy treatments performed on lung tumors. Another nonlimiting example is dual energy fluoroscopy for markerless motion tracking of lung tumors. Because the soft tissue-enhanced image enables a tumor to be visualized better by eliminating imaging of the bone, a computer can be more readily utilized to track the tumor.

A technical effect of the invention is the ability to acquire a highlighted or enhanced image of soft tissue while potentially decreasing the number of exposures to radiation conventionally associated with dual-energy imaging techniques, and without certain hardware and processing demands that have been associated with MTNN techniques.

Other aspects and advantages of this invention will be better appreciated from the following detailed description.

### BRIEF DESCRIPTION OF THE DRAWINGS

FIG. 1 is a flowchart showing a dual-energy imaging technique for highlighting or enhancing the visualization of soft tissues by suppressing the imaging of hard tissues.

FIG. 2 shows two radiographic images (left and center) produced at, respectively, relatively high and low energies, and a third image (“DE Soft”) generated by applying a logarithmic subtraction algorithm.

FIG. 3 is a flowchart showing a dual-energy imaging technique for highlighting or enhancing the visualization of soft tissues in accordance with a nonlimiting embodiment of the present invention.

FIG. 4 shows radiographic images produced by the dual-energy imaging technique of FIG. 3.

### DETAILED DESCRIPTION OF THE INVENTION

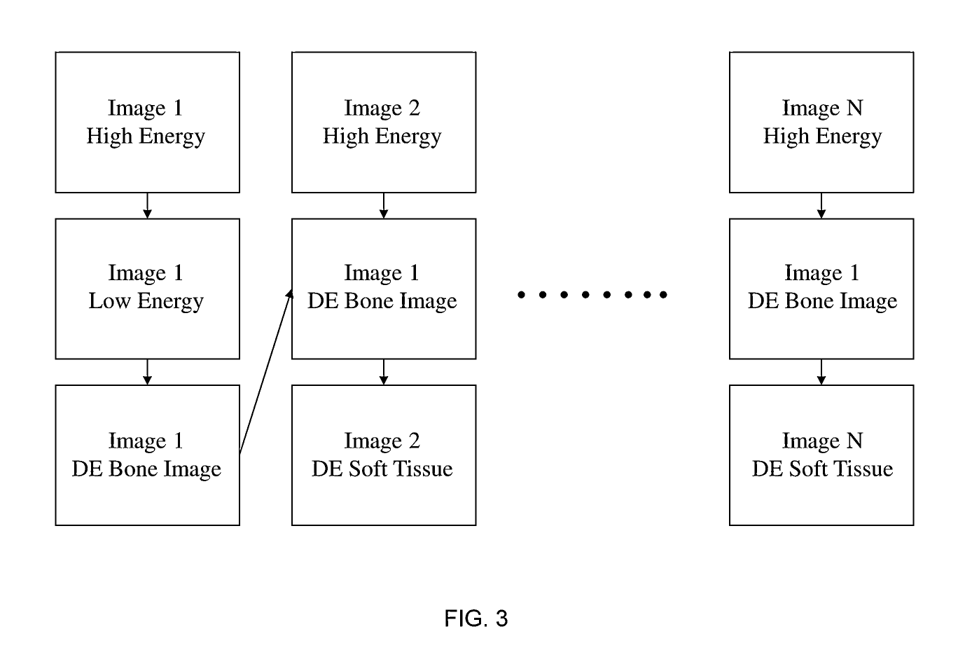

FIG. 3 represents a flowchart showing steps of a dual-energy (DE) imaging technique capable of highlighting or enhancing the visualization of soft tissues, particularly with respect to a nonlimiting embodiment of the present invention. The dual-energy imaging technique can be used to enhance the visualization of soft tissues by suppressing images of hard tissue in a radiograph. As used herein, “soft tissues” and “hard tissues” are relative terms used in reference to each other. In particular applications in which a soft tissue of particular interest is a tumor, “hard tissues” will typically encompass bones and “soft tissue” will encompass all tissues other than bones. As a particular example, dual-energy imaging techniques described herein can be used to enhance the visualization of a lung tumor in a patient by suppressing images of ribs that might otherwise obscure the image of the tumor. As will also be described below, such a capability is not only advantageous for diagnostic purposes, but can also be advantageous when treating a patient, such as when a patient receives image-guided radiotherapy (IGRT).

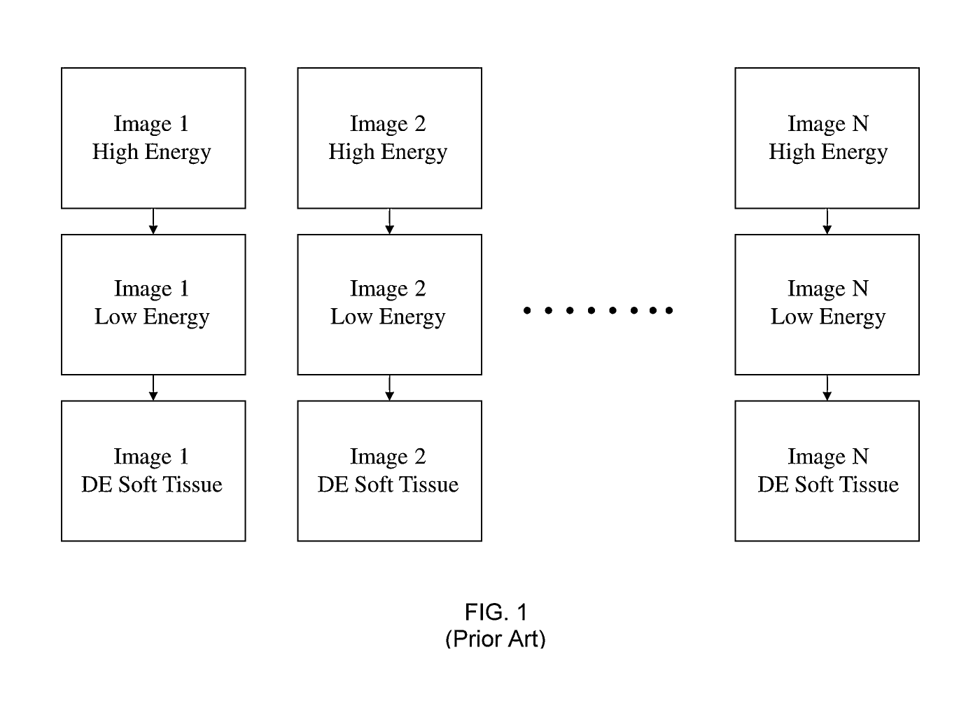

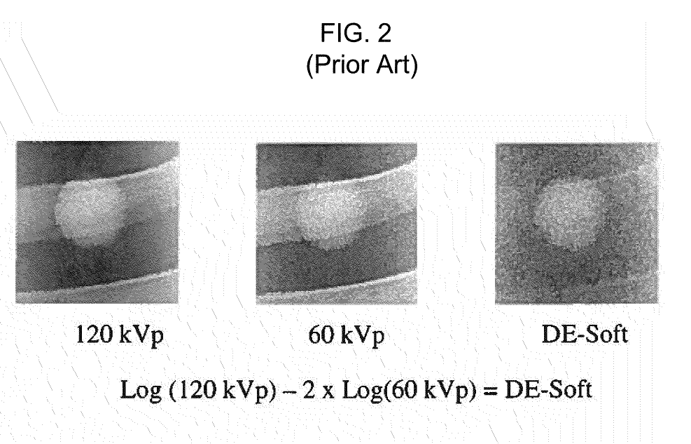

As previously discussed, the DE imaging technique represented by FIGS. 1 and 2 generally requires the acquisition of at least a high-energy radiographic image (“Image 1 High Energy”) and a low-energy radiographic image (“Image 1 Low Energy”), which are then used to perform weighted subtraction to create a third image (“Image 1 DE Soft Tissue”) in which soft tissues are highlighted or enhanced. FIG. 3 represents a flowchart corresponding to what may be termed a serial DE imagining technique, and similarly involves the acquisition of at least a high-energy radiographic image (“Image 1 High Energy”) and a low-energy radiographic image (“Image 1 Low Energy”), but which are then used to perform weighted subtraction to create a third image (“Image 1 DE Bone Image”) in which bones (or other hard tissues) can be highlighted or enhanced. As such, whereas weighted subtraction in FIG. 1 yields a highlighted or enhanced image of soft tissues (“Image 1 DE Soft Tissue”), weighted subtraction in FIG. 3 yields an image (“Image 1 DE Bone Image”) in which imaging of hard tissues is highlighted or enhanced, whereas imaging of soft tissues is suppressed. Such an image may be referred to herein as a “bone-weighted image” or, more generically, a “hard tissue-enhanced image.” As evident from FIG. 3, this bone-weighted image, which can be acquired at any time during a patient's care, preferably at or near the beginning thereof, is thereafter available for use throughout the course of additional radiography procedures performed on the patient (“Image 2” through “Image N” in FIG. 3), including but not limited to therapeutic treatments performed on the patient. In particular, FIG. 3 represents the bone-weighted image, “Image 1 DE Bone Image,” as being utilized in each subsequent radiography procedure, during which a second high-energy radiographic image (“Image 2 High Energy” through “Image N High Energy”) is acquired, but not a second low-energy radiographic image. One or more soft tissue images (“Image 2 DE Soft Tissue” through “Image 2 DE Soft Tissue”), in which soft tissues are highlighted or enhanced and images of bones (or other hard tissues) are suppressed, can then be created by performing weighted subtraction between the “Image 2 High Energy” image and the “Image 1 DE Bone Image.” Such an image may be referred to herein as a “soft tissue-enhanced image.”

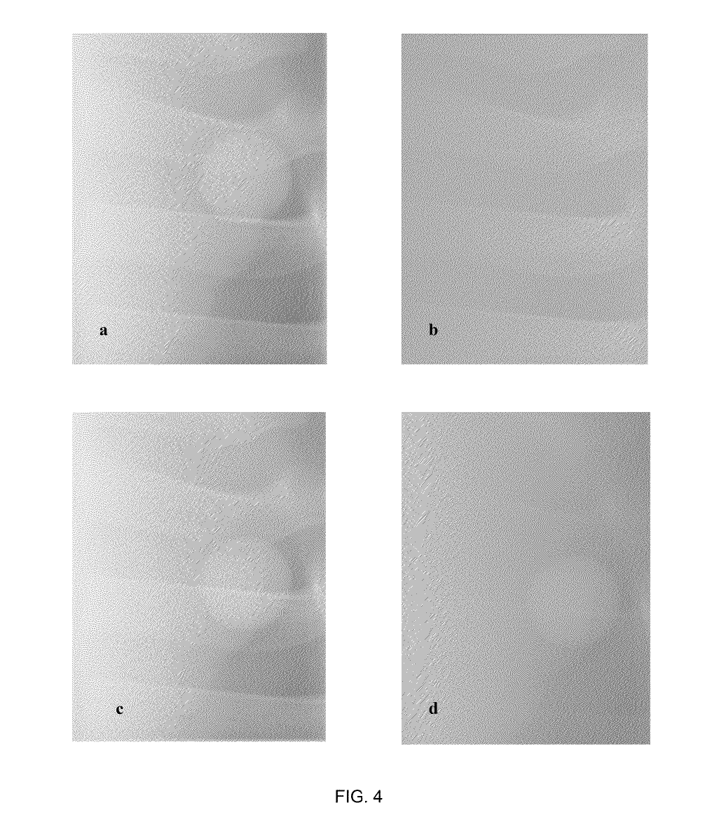

In investigations leading to the procedure represented in FIG. 3, a Quasar motion phantom (Modus Medical Devices Inc, London, Ontario) was modified by affixing pork ribs to its platform. A cedar insert with a small spherical volume was used to simulate a lung and tumor, respectively. As a baseline, serial images (Images 1−N) were obtained at various phases simulating respiratory motion. At each respiratory phase, both 120 kVp (“high” energy) and 60 kVp (“low” energy) radiographic images were obtained, and a weighted logarithmic subtraction algorithm (log(120 kVp)-2×log(60 kVp)) was performed to create rib-suppressed images, corresponding to the enhanced images of soft tissues (“Image 1 DE Soft Tissue”) represented by the prior art method of FIG. 1. An example of one of the high-energy images is depicted as image “a” in FIG. 4.

As an experimental alternative, a bone-weighted image was acquired with the use of the initial high-energy and low-energy DE images (Image 1). In particular, the bone-weighted image was obtained by performing a weighted logarithmic subtraction algorithm with the initial high-energy and low-energy DE images (log(120 kVp)-4×log(60 kVp)) to suppress soft tissue-imaging and enhance bone imaging, such that the bone-weighted image corresponded to the “Image 1 DE Bone Image” of FIG. 3. The bone-weighted image is depicted as image “b” in FIG. 4, and was subsequently applied to subsequent high-energy (120 kVp) images (Images 2−N) to produce rib-suppressed images at each respiratory phase. An example of one of the subsequent high-energy images is depicted as image “c” in FIG. 4. The contrast-to-noise ratio (CNR) of the simulated tumor was compared at each respiratory phase for both techniques to assess tumor visibility.

The baseline and experimental techniques resulted in complete subtraction of the ribs (rib suppression) providing enhanced visibility of the simulated tumor. An example of one of the rib-suppressed images produced by the experimental technique is depicted as image “d” in FIG. 4, and evidences that the simulated tumor is visible and not obscured by images of the ribs. Comparison of the experimental and baseline techniques showed no difference in CNR, indicating the experimental technique may be an efficient method of rib suppression.

It was further concluded that such a capability could be suitable for use with radiotherapy patients, for whom serial and fluoroscopic images are often obtained. In particular, radiotherapy patients often receive 5-30 fractions of radiation, and hence are imaged multiple times. With the experimental technique described above, for each radiograph step, acquisition of only a single image would be required for the experimental technique, as opposed to the acquisition of two images (high and low energy) required for the baseline technique.

An exemplary procedure implementing the experimental procedure outlined above would generally follow the flowchart of FIG. 3. More particularly, steps would include obtaining at least relatively high- and low-energy radiographic images (for example, 120 kVp and 60 kVp) of a patient in the vicinity of a tumor (or other soft tissue) of interest, and then perform a weighted subtraction algorithm of the high- and low-energy images to obtain a bone-enhanced image, essentially is indicated in the lefthand column of FIG. 3. Bone-enhanced images may be obtained from high- and low-energy images acquired at any and all gantry angles, as may be the prerogative of the healthcare provider. A weighted subtraction algorithm may be performed on the same high- and low-energy images to obtain a soft tissue-enhanced image, essentially is indicated in the lefthand column of FIG. 1. On any subsequent patient visit, only a single (preferably high-energy) radiographic image need be obtained for the patient, which can then be aligned with the previously-obtained bone-enhanced image. A weighted subtraction of the bone-enhanced image can then be performed with the single image to obtain a soft tissue-enhanced image, as indicated in the center and righthand columns of FIG. 3. Theoretically, the same bone-enhanced image could be used throughout a series (1 . . . N) of imaging procedures performed on a patient to evaluate and/or treat a tumor.

As a possible alternative to the above, it is foreseeable that a bone-enhanced image could be created using computed tomography (CT) data obtained at the time of radiotherapy treatment planning. A digitally reconstructed radiograph (DRR) could then be produced for each gantry angle considered, and the DRR used in place of the bone-enhanced image or to augment the use of the bone-enhanced image, for example, to remove additional anatomical clutter from the soft tissue-enhanced image.

From the foregoing, it should be appreciated that dual energy imaging techniques as described above are capable of providing bone suppression for serial images in which a bone (or other hard tissue) image is obtained from a dual energy image set, and then applied to one or more subsequent images. Because comparisons of the experimental and baseline techniques described above showed no significant difference in CNR, the experimental technique may be an efficient method of rib suppression. This aspect can be utilized for diagnostic purposes as well as treatments of soft tissue, and particularly patients receiving image-guided radiotherapy (IGRT), in which case an enhanced soft-tissue image acquired through the technique may be used to reduce the unnecessary irradiation of tissue surrounding a tumor by avoiding imaging discrepancies that can arise from the prior practice of requiring two separate energy images at each instance, which can lead to image discrepancies attributable to movement of the patient.

In addition, the technique described herein can be used in conjunction with fluoroscopy to perform frame-by-frame subtraction of hard tissue images to enhance visibility of a tumor in real time and may enable real-time tumor motion tracking. In the past, whereas dual energy imaging has been shown to improve the visualization of lung tumors on planar kV x-ray images, and combining dual energy imaging with fluoroscopy could enable real-time tumor motion tracking, such an approach would require a “fast switching” x-ray generator to produce alternating high- and low-energy x-ray images. Because such an approach may be impractical for various reasons, an investigation was devised in which dual energy images would be obtained prior to intra-fraction imaging in a manner similar to what has been described above and represented in the lefthand column of FIG. 3. Briefly, 60 kVp and 120 kVp image sets would be obtained at the same respiratory phase, and a subtraction algorithm would be performed to produce a bone-weighted image that (similar to the bone-weighted image “b” in FIG. 4) highlight/enhances imaging of ribs and other skeletal bones, while suppressing surrounding soft tissue that included a tumor. A fluoroscopic image sequence (120 kVp, 1.5 mA) would then be obtained, and a second subtraction algorithm performed on a frame-by-frame basis using the previously obtained bone-weighted image. This subtraction algorithm would provide for a real-time fluoroscopic image sequence in which the ribs are suppressed, providing improved visualization of a tumor.

To evaluate this procedure and its algorithm, both 60 kVp and 120 kVp fluoroscopic image sets were obtained on three patients (for a total of nine fluoroscopic sets) using an on-board imager. As a baseline procedure, these images were aligned based on phase from the respiratory signal, and frame-by-frame subtraction was performed to produce a dual energy soft tissue fluoroscopic image set. As part of an experimental procedure, a logarithmic subtraction algorithm was separately performed on the first 60 and 120 kVp image pair to produce a bone-weighted image that was subsequently used to perform weighted subtraction of the bone image from the 120 kVp image of each image set. For this purpose, a template-based motion-tracking algorithm was used on both image sequences and the tracking coordinates, and peak-to-side lobe ratios (PSR) were compared on a frame-by-frame basis. A total of 1036 fluoroscopic imaging frames were compared, from which no significant difference was identified in the location of the tumor center using that baseline and experimental fluoroscopy procedures. The average differences in the tumor centroid coordinates were 0.01±0.67 mm and 0.12±0.89 mm in the x and y directions, respectively. The PSR values were also comparable with average values of 5.25±3.13 and 5.32±2.64 (p=0.22) for the baseline and experimental fluoroscopy procedures, respectively. From these results, it was concluded that the experimental approach would be capable of providing improved visualization of lung tumors (and other soft tissue) on fluoroscopic imaging without the need for additional or specialized hardware such as a fast-switching x-ray generator. Moreover, the experimental procedure would be capable of providing real-time motion tracking of lung tumors, providing results that can be comparable to conventional dual-energy subtraction fluoroscopy, for example, corresponding to the baseline procedure reported above.

While the invention has been described in terms of specific embodiments and investigations, it is apparent that other forms could be adopted by one skilled in the art. For example, while particular logarithmic subtraction algorithms were used in investigations reported herein, other suitable algorithms could be devised and used if the ultimate effect is to either enhance or suppress, as the case may be, soft tissue (e.g., tumor) or hard-tissue (e.g., bone) images. Therefore, the scope of the invention is to be limited only by the following claims.

1. An imaging process for selective visualization of soft tissues of a body, the process comprising:
producing a hard tissue-enhanced image of a body containing both soft and hard tissues, the hard tissue-enhanced image containing images of the hard tissues that are enhanced relative to the soft tissues;
obtaining a radiographic image of the body and the soft and hard tissues thereof; and
performing a weighted subtraction algorithm between the radiographic image and the hard tissue-enhanced image to produce a soft tissue-enhanced image in which imaging of the soft tissues is enhanced relative to the hard tissues.

2. The imaging process according to claim 1, wherein the hard tissue-enhanced image is produced by:
obtaining initial radiographic images of the body and the soft and hard tissues thereof, a first of the initial radiographic images being at a first energy level and a second of the initial radiographic images being at a second energy level that is lower than the first energy level; and then
performing a weighted subtraction algorithm on the first and second initial radiographic images to produce the hard tissue-enhanced image.

3. The imaging process according to claim 2, wherein the radiographic image is obtained at the first energy level.

4. The imaging process according to claim 1, wherein the weighted subtraction algorithm performed between the radiographic image and the hard tissue-enhanced image is a logarithmic subtraction algorithm.

5. The imaging process according to claim 1, wherein the soft tissues comprise a tumor.

6. The imaging process according to claim 5, wherein the body comprises lungs of a patient and the tumor is located in the lungs.

7. The imaging process according to claim 1, wherein the process is performed in combination with an image-guided radiotherapy procedure.

8. The imaging process according to claim 7, wherein the radiographic image is one of a plurality of radiographic images, and the weighted subtraction algorithm is performed between the hard tissue-enhanced image and each of the plurality of radiographic images to produce a corresponding plurality of the soft tissue-enhanced images in a real-time fluoroscopic image sequence.

9. An imaging process for selective visualization of soft tissues of a body, the process comprising:
obtaining radiographic images of a body containing both soft and hard tissues, a first of the radiographic images being at a first energy level and a second of the radiographic images being at a second energy level that is lower than the first energy level;
performing a first weighted subtraction algorithm on the first and second radiographic images to produce a hard tissue-enhanced image in which imaging of the hard tissues is enhanced relative to the soft tissues;
obtaining a third radiographic image of the body and the soft and hard tissues thereof; and then
performing a second weighted subtraction algorithm between the third radiographic image and the hard tissue-enhanced image to produce a soft tissue-enhanced image in which imaging of the soft tissues is enhanced relative to the hard tissues.

10. The imaging process according to claim 9, wherein the first energy level is about 120 kVp and the second energy level is about 60 kVp.

11. The imaging process according to claim 9, wherein the first and second weighted subtraction algorithms are different logarithmic subtraction algorithms.

12. The imaging process according to claim 9, wherein the soft tissues comprise a tumor.

13. The imaging process according to claim 12, wherein the body comprises lungs of a patient and the tumor is located in the lungs.

14. The imaging process according to claim 9, wherein the process is performed in combination with an image-guided radiotherapy procedure to treat a tumor or a fluoroscopy procedure for markerless motion tracking of a tumor.

15. The imaging process according to claim 14, wherein the third radiographic image is one of a plurality of subsequent radiographic images, and the second weighted subtraction algorithm is performed between the hard tissue-enhanced image and each of the plurality of subsequent radiographic images to produce a corresponding plurality of the soft tissue-enhanced images in a real-time fluoroscopic image sequence.

16. An imaging process in combination with an image-guided radiotherapy procedure to treat a tumor or a fluoroscopy procedure for markerless motion tracking of a tumor, the process comprising:
obtaining radiographic images of a body containing hard tissues that include bones and containing soft tissues that include a tumor, a first of the radiographic images being at a first energy level and a second of the radiographic images being at a second energy level that is lower than the first energy level;
performing a first weighted subtraction algorithm on the first and second radiographic images to produce a bone-enhanced image in which imaging of the bones is enhanced relative to the tumor;
obtaining a series of subsequent radiographic images of the body including the tumor, each of the series of subsequent radiographic images being at the first energy level; and
performing a second weighted subtraction algorithm between the bone-enhanced image and each of the series of subsequent radiographic images to produce a corresponding plurality of soft tissue-enhanced images in a real-time fluoroscopic image sequence, wherein each of the plurality of soft tissue-enhanced images contains imaging of the tumor that is enhanced relative to the bones.

17. The imaging process according to claim 16, wherein the first energy level is about 120 kVp and the second energy level is about 60 kVp.

18. The imaging process according to claim 16, wherein the first and second weighted subtraction algorithms are different logarithmic subtraction algorithms.

19. The imaging process according to claim 16, wherein the body comprises lungs of a patient and the tumor is located in the lungs.

20. The imaging process according to claim 19, wherein the series of subsequent radiographic images are obtained a different phases of respiratory motion.
